(19) United States
(12) Patent Application Publication
(10) Pub. No.: US 2025/0268263 A1
(43) Pub. Date: Aug. 28, 2025

(54) ENTOMOPATHOGENIC FUNGI CONIDIA POWDER AND THE PRODUCTION METHOD THEREOF

(71) Applicant: Taichung District Agricultural Research and Extension Station, Ministry of Agriculture (TW), Dacun Township (TW)

(72) Inventor: Pei-Hsin Lo, Dacun Township (TW)

(21) Appl. No.: 19/034,818

(22) Filed: Jan. 23, 2025

(30) Foreign Application Priority Data
Feb. 22, 2024 (TW) 113106429

(51) Int. Cl. A01N 63/30; A01N 25/12; A01P 7/04; C12N 1/14; C12N 3/00

(52) U.S. Cl. CPC A01N 63/30; A01N 25/12; A01P 7/04; C12N 1/14; C12N 3/00

(57) ABSTRACT

The present disclosure provides an entomopathogenic fungi conidia powder, which comprises a powder and conidia attached to the surface of the powder. On the other hand, the present disclosure further provides a method for producing the entomopathogenic fungi conidia powder. Based on the disclosure of the present disclosure, the production method uses a powder to adhere to the entomopathogenic fungi conidia, and allowing them to be stored at room temperature for at least 7 months with a substantially same amount of conidia population. In addition, the production method using solid fermentation reduces production costs.

### CROSS-REFERENCE TO RELATED APPLICATION

This application claims the benefit of Taiwan Utility Application No. 113106429, filed on Feb. 22, 2024, which is incorporated herein by reference in its entirety.

### FIELD OF THE INVENTION

The present disclosure relates to a microbial insecticide, and more particularly to an entomopathogenic fungal conidia powder and the production method thereof.

### BACKGROUND OF THE INVENTION

Climate change has led to rising global temperatures and an increase in the frequency and severity of pest and disease outbreaks, such as those caused by Tessaratoma papillosa (lychee stink bug), Spodoptera frugiperda (fall armyworm) and rice blast disease (caused by Magnaporthe oryzae). Farmers have to spray more chemical pesticides and more often with a view to preventing pest diseases. On the other hand, increasingly great attention is attached to the development and use of biopesticides to address issues like chemical pesticide residues and environmental protection. Biopesticides can be categorized into microbial pesticides, pesticides made from natural materials, and biochemical pesticides. The active ingredient of the microbial pesticides is either a living microorganism or the secretions it produces, offering insecticidal, bactericidal, and herbicidal functions, with additional advantages such as high safety and environmental friendliness. Unlike chemical pesticides, microbial pesticides do not remain in crops and thus do not affect human beings and environments. Furthermore, microbial pesticides help mitigate the environmental damage and soil degradation often associated with chemical pesticides.

Microbial pesticides comprise microbial insecticides and microorganism bactericides. Currently, most commercially available microbial insecticides are based on Bacillus thuringiensis, with fewer products utilizing entomopathogenic fungi. Therefore, the development of microbial insecticides other than Bacillus thuringiensis could provide greater diversity in this field. Entomopathogenic fungi are fungi that parasitize insects (i.e. the hosts). While different species of entomopathogenic fungi infect different types of insects, they all infect insects through conidia. Once germinated, the conidia form hyphae that intrude into the bodies of insects and reproduce massively, and eventually cause the insects to die within 3 to 14 days after being parasitized. Although entomopathogenic fungi can infect most insect species, entomopathogenic fungi do not have any adverse effects on human beings or livestock. Therefore, entomopathogenic fungi are suitable for use in manufacturing microbial insecticides. Furthermore, under favorable environmental conditions, entomopathogenic fungi can remain in farmlands, spreading and continuously infecting other insects.

However, the active ingredient of the microbial pesticides is typically a living microorganism (for example, entomopathogenic fungal conidia) or the secretions it produces. Thus, related products currently have relatively short shelf lives and require cold storage to maintain the activity of the microorganisms. Therefore, it is important to develop a microbial insecticide that can be preserved at the room temperature for a long period of time and exhibits strain diversity.

### SUMMARY OF THE INVENTION

To meet the needs facing this field, the present disclosure provides an entomopathogenic fungal conidia powder and the production method thereof, producing conidia through solid-state fermentation, allowing a powder to adhere to the conidia cultured, reducing cost and extending shelf life.

To achieve the above propose, the present disclosure provides a method of producing an entomopathogenic fungal conidia powder, comprising: (a) cultivating the entomopathogenic fungi with a solid culture medium through a solid-state culture to produce conidia; (b) adding a powder to adhere to the conidia; and (c) sieving to obtain the conidia powder.

Preferably, in the method, the solid culture medium comprises rice and soy protein.

Preferably, in the method, the powder is added when the water content of the solid culture medium is 30-40 wt %, and more preferably 36-39 wt %.

Preferably, in the method, the temperature of the solid-state culture is 26-32° C., and more preferably 28° C.

Preferably, in the method, the solid-state culture is tray-style solid-state fermentation.

Preferably, in the method, the sieving is performed with a sieve with a mesh size of 14-30, and more preferably a mesh size of 20.

Preferably, in the method, the powder is 5-40% (w/w), more preferably 10-30% (w/w), and even more preferably 20% (w/w), of the weight of the solid culture medium. Preferably, in the method, a step of drying the conidia is excluded.

In another aspect of the present disclosure, provided herein is an entomopathogenic fungal conidia powder, comprising a powder and conidia attached to a surface of the powder.

Preferably, in the entomopathogenic fungal conidia powder, the entomopathogenic fungi comprise Beauveria spp., Metarhizium anisopliae, Metarhizium rileyi, Isaria javanica, and Purpureocillium takamizusanense.

Preferably, in the entomopathogenic fungal conidia powder, the powder comprises a thickening agent. In an embodiment of the present disclosure, the thickening agent comprises: Gum Arabic, Citrus Pectin, Hydroxypropyl Starch, Sodium Polyacrylate, Carrageenan, Gellan Gum, Hydroxyethyl Cellulose, Pullulan, Carboxymethyl Cellulose, Methylcellulose, Xanthan Gum, Guar Gum and Sodium Alginate.

Preferably, the entomopathogenic fungal conidia powder is produced by the any one of aforementioned method.

### BRIEF DESCRIPTION OF THE DRAWINGS

FIG. 1 shows a schematic view of (A) tray-style solid-state fermentation, (B) sealed-bag solid-state fermentation, and (C) a 5-liter solid-state fermentation tank.

FIG. 2 shows a schematic view of (A) tray-style solid-state fermentation, (B) addition of a thickening agent to adhere to the conidia followed by sieving, and (C) sieved conidia powder.

FIG. 3 shows a comparison of the shelf life of the conidia powder at different temperatures.

FIG. 4 shows a comparison of the shelf life of the conidia powder produced with different techniques.

### DETAILED DESCRIPTION

Unless otherwise defined, all technical and scientific terms used herein have the same meaning as commonly understood by one of ordinary skill in the art. In case of a conflict, definitions specified herein shall prevail.

The unit symbols “wt %” and “% (w/w)” as used herein refer to mass percent concentration, and are defined as the percentage obtained by dividing the mass of the solute by the mass of the corresponding mixture (inclusive of the mass of the solute).

The expression “produced from” as used herein has the same meaning as the term “comprising”. The terms “includes, including,” “comprises, comprising,” “has, having,” and “contains, containing” used herein, or any other variants, are intended to be non-exclusive and all-encompassing. For instance, a composition, process, method, product or device that contains a plurality of elements listed in a checklist does not necessarily contain only the elements listed in the checklist but may also comprise any other elements that are not definitely listed in the checklist but are intrinsic to the composition, process, method, product or device. In general, the verb “include” used herein and the verb “comprise” are interchangeable, allowing the existence of one or more other features or ingredients.

The indefinite articles “a” and “an” as used herein and preceding elements or ingredients disclosed herein are intended to provide a non-restrictive description of the quantity (i.e., the number) of the elements or ingredients disclosed herein. Therefore, the indefinite articles “a” and “an” shall be interpreted to mean “comprising one or at least one,” and the singular form of the elements or ingredients may also be interpreted to mean “comprising a plurality of,” unless the number obviously refers to an odd number.

The propose of the present disclosure is to provide a method of producing an entomopathogenic fungal conidia powder, comprising: (a) cultivating the entomopathogenic fungi with a solid culture medium through a solid-state culture to produce conidia; (b) adding a powder to adhere to the conidia; and (c) sieving to obtain the conidia powder.

The terms “solid-state culture” and “solid-state fermentation” as used herein are interchangeable and refer to the process in which entomopathogenic fungi not only produce conidia but also grow massively under appropriate culture conditions in the absence of any liquid-state culture medium.

The terms “conidium” and “conidia” as used herein refer to asexual spores produced by entomopathogenic fungi. These spores represent an important method of reproduction for entomopathogenic fungi and facilitate their spread to new environments. The conidia may be unicellular or multicellular, and their morphology varies among different entomopathogenic fungi, including ovoid, fusoid, pyriform, filiform, obpyriform, and falcate. Moreover, the surface texture of the conidia exhibits various patterns, such as punctate, reticulate, echinate, and verrucose.

In an embodiment, the solid culture medium comprises rice and soy protein. The term “solid culture medium” as used herein refers to a culture medium that enhances the reproduction and growth of entomopathogenic fungi and provides a carbon source, nitrogen source or growth factor required for the growth. Based on their composition, solid culture media can be classified into synthetic media and natural media. Synthetic media are characterized by definite chemical ingredients, comprising: Lysogeny broth (LB broth), yeast extract, Sabouraud's agar, and potato dextrose Agar. Natural media contain any ingredients (for example, fruits, vegetables, cereals, beans, dairy products, and meat) which provide nutrients for entomopathogenic fungi.

In an embodiment, the powder is added when the water content of the solid culture medium is 30-40 wt %, and preferably 36-39 wt %. The term “water content” as used herein refers to mass water content, which is the percentage obtained by dividing the mass of water by the mass of the corresponding mixture (including mass of water).

In an embodiment, the temperature of the solid-state culture is 26-32° C., and preferably 28° C. The temperature range disclosed herein can be applicable to any devices capable of controlling temperature and well known among in the art, including water baths and incubators.

In an embodiment, the solid-state culture is tray-style solid-state fermentation. The term “tray-style solid-state fermentation” as used herein refers to solid-state fermentation that takes place in any culture dishes which are of any sizes and shapes and are made of any materials. The culture dishes include plastic/glass Petri dishes, stainless steel dishes, iron dishes, plastic dishes or ceramic dishes. The method of the present disclosure is conducive to increasing the contact surface area between the solid culture medium and the entomopathogenic fungi and increasing entomopathogenic fungi culture yield.

In an embodiment, the sieving is performed with a sieve with a mesh size of 14-30, and preferably a mesh size of 20. The terms “mesh” and “mesh number” as used herein are interchangeable and refer to the number of openings per linear inch of a sieve. The relationship between “mesh number” and “mesh size” is expressed by the equation: mesh number×mesh size (μm)=15,000. Therefore, a sieve of the present disclosure may also refer to a pore diameter ranging from approximately 1070 μm to 500 μm, and more preferably 500 μm. It should be noted that the pore diameter of the sieve used in this application should be adjusted accordingly to match the specific solid culture medium being used. For instance, a solid culture medium with smaller particle sizes compared to white rice, the sieve pore diameter used for sieving should be capable of accommodating a range smaller than 1070 μm to 500 μm.

In an embodiment, the powder accounts is 5-40% (w/w), preferably 10-30% (w/w), and more preferably 20% (w/w), of the weight of the solid culture medium.

In an embodiment, a step of drying the conidia is excluded. Unlike any other conventional techniques, the method of the present disclosure does not obtain the conidia powder by means of water-washing. Therefore, the conidia powder obtained with the method of the present disclosure is unlikely to contain any traces of water, and thus the method of the present disclosure does not require performing any drying procedure. The drying method may include: sun drying, air-dry, shade-dry, heat drying, spray drying, vacuum drying, or freeze-drying.

The present disclosure also provides an entomopathogenic fungal conidia powder, comprising a powder and conidia attached to a surface of the powder.

In an embodiment, in the entomopathogenic fungal conidia powder, the entomopathogenic fungi comprise Beauveria spp., Metarhizium anisopliae, Metarhizium rileyi, Isaria javanica, and Purpureocillium takamizusanense.

The term “entomopathogenic fungi” as used herein refers to a type of fungus that parasitizes the larvae or adults of insects, spiders, or similar organisms. Different entomopathogenic fungi infect different types of insects. When the spores come into contact with the surface of the body of an insect, they germinate and invade into the body of the insect, eventually leading to the death of the insect. The term “entomopathogenic fungi” as used herein does not refer specifically to a specific monophyletic group, but may refer generally to Hypocreales, Paecilomyces, Hirsutella, Metarhizium, Nomuraea, Cordyceps, Entomophthora, Zoophthora, Pandora, and Entomophaga.

In an embodiment, in the entomopathogenic fungal conidia powder, the powder comprises a thickening agent. In a preferred embodiment, the thickening agent comprises Gum Arabic, Citrus Pectin, Hydroxypropyl Starch, Sodium Polyacrylate, carrageenan, gellan gum, Hydroxyethyl Cellulose, Pullulan, Carboxymethyl Cellulose, Methylcellulose, Xanthan Gum, Guar Gum and Sodium Alginate.

In an embodiment, the entomopathogenic fungal conidia powder is produced by any of aforementioned method.

The materials, method and examples disclosed herein are merely illustrative. Unless otherwise specified, the materials, method and examples disclosed herein are not intended to be restrictive of the present disclosure but are appropriate method, materials and examples, even though the present disclosure can be implemented or tested with similar or equivalent methods and materials.

### Example 1: Solid-State Fermentation Techniques

The present disclosure discloses three different solid-state fermentation techniques: tray-style fermentation, sealed-bag fermentation and fermentation tank fermentation. Compared with liquid-state fermentation, solid-state fermentation has advantages as follows: solid-state fermentation enables a conidia population to grow at a high density, which means that a larger conidia population can be cultured in a culture medium of a fixed volume through solid-state fermentation, and the product (conidia) can be separated from the culture medium easily, for example, through water-washing, to obtain the conidia, or by using a powder to adhere to the conidia.

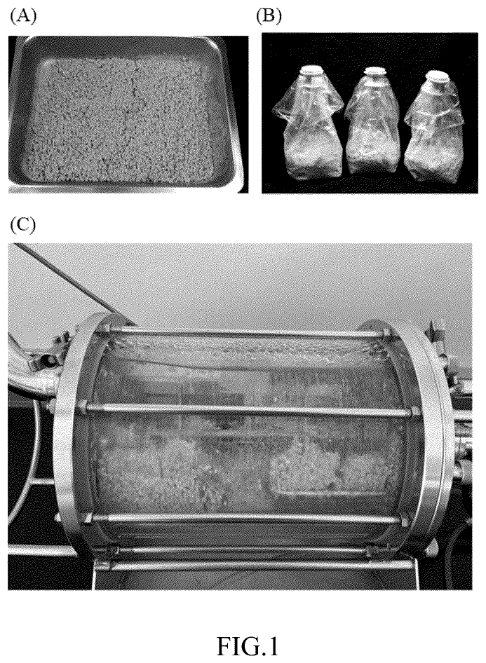

Tray-style solid-state fermentation (FIG. 1(A)) enables a culture medium (for example, rice) to have a relatively large fermentation surface area and thereby increases the conidia culture yield to be greater than >1010 spores/g. Sealed-bag solid-state fermentation (FIG. 1(B)) entails placing rice in a sealed-bag to undergo fermentation to attain the conidia culture yield of around 10⁹ spores/g. Solid-state fermentation tank (FIG. 1(C)) also achieves the conidia culture yield of 10⁹ spores/g but incurs high cost and may end up with accumulation of water at the tank bottom.

All the three solid-state fermentation techniques are effective in producing a large amount of conidia, because tray-style solid-state fermentation is simple and convenient to operate, incurs low equipment cost, and merely requires any dishes capable of containing a culture medium. Therefore, all experiments disclosed in the present disclosure below use tray-style solid-state fermentation as an example of solid-state fermentation techniques.

### Example 2: Tray-Style Solid-State Fermentation Process for Entomopathogenic Fungi

Preparation of an inoculum of entomopathogenic fungi: the fermentation process is started with a conidia suspension of entomopathogenic fungi (including Beauveria spp., Metarhizium anisopliae, Metarhizium rileyi, Isaria javanica and/or Purpureocillium takamizusanense) of a concentration of 1.5 ml 2×10⁹ spores/ml in 150 ml of potato dextrose broth (Table 1) contained in a 250 ml grooved Erlenmeyer flask. Upon completion of the inoculation, the 250 ml grooved Erlenmeyer flask is placed in an incubator at 28° C. and shaken at 150 rpm to undergo dark cultivation for 5 days in order to obtain the inoculum for use in solid-state fermentation.

TABLE 1
formula of potato dextrose broth
Potato dextrose broth (1 liter)

| | | |
|---|---|---|
| Potato extract | 4.0 | g |
| Dextrose | 20.0 | g |
| Water | 1000 | ml |

Solid substrate preparation: after being rinsed, the rice is immersed in water, left to stand at 4° C. for 24 hours, filtered with a filtering net, left to stand for 30 minutes, followed by the introduction of soy protein, evenly mixed, subjected to high-temperature, high-pressure sterilization (121° C., 20 min), cooled, and subjected to entomopathogenic fungal inoculation.

Entomopathogenic fungal inoculation: a 15% (w/v) inoculum of entomopathogenic fungi is inoculated on a prepared solid substrate and evenly mixed.

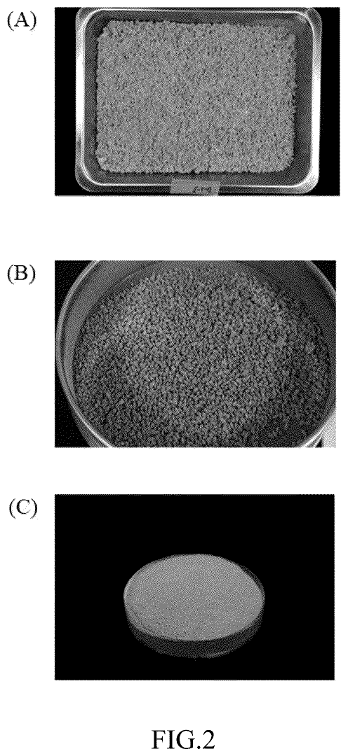

Entomopathogenic fungi solid-state fermentation: as shown in FIG. 2(A), the rice for use in inoculation of entomopathogenic fungi is left to stand and cultured in an incubator at 28° C., cultured without being illuminated for 2 days, placed in a stainless steel dish and laid flat therein with a depth of around 2 to 3 cm, and cultured in an incubator at 28° C. without being illuminated for 10 to 12 days to undergo solid-state fermentation of entomopathogenic fungal conidia, producing a large amount of conidia on the surfaces of the rice. Upon completion of solid-state fermentation, the rice has water content of 36 wt %-39 wt %, and thus a powder can be directly added, allowing different entomopathogenic fungal conidia powder to be prepared subsequently.

### Example 3: Preparation of Entomopathogenic Fungal Conidia Powder

Upon completion of tray-style solid-state fermentation in example 2, a large amount of entomopathogenic fungal conidia are produced on the surfaces, using a powder to adhere to the conidia. First, a test of adhesion effect is performed with Pullulan, which is a polysaccharide and possesses satisfactory attachment capability. Upon completion of entomopathogenic fungi solid-state fermentation, 10-20% (w/w) Pullulan powder is added and evenly mixed. Then, Pullulan enables the conidia to be adhered from the surface of the rice, and the rice is separated from a spore powder with a sieve with a mesh size of 20 (FIG. 2(B)), and sieving to obtain the conidia powder (FIG. 2(C)).

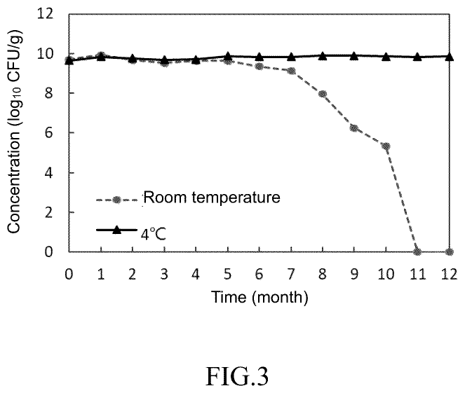

The conidia powder obtained is compared in terms of their shelf life at different temperatures. As shown in FIG. 3, the conidia powder obtained can be preserved at room temperature for 7 months with a substantially same amount of conidia population. However, by the end of the 9th month, the conidia population decreases to 102 CFU/g. In contrast, when the conidia powders preserved at 4° C., the conidia population remains stable for at least one year with no significant changes. Therefore, the conidia powder can serve as a raw material for preparing formulations such as wettable powders and water-dispersible granules.

The results above show that the powder with adhesion capability can serve as options for adhering to conidia. Therefore, the present disclosure further tests various powders as follows: Gum Arabic, Citrus Pectin, Hydroxypropyl Starch, Sodium Polyacrylate, carrageenan, gellan gum, Hydroxyethyl Cellulose, Pullulan, Carboxymethyl Cellulose, Methylcellulose, Xanthan Gum, Guar Gum and/or Sodium Alginate. Although the adhesion effects vary among these powders, all of them can adhere to most conidia, making them viable options for the present disclosure.

### Example 4: Comparison of the Shelf Live Using Different Techniques for Preparing Conidia Powder

In general, conventional conidia powder production methods involve using a water-washing technique to separate conidia from solid-state fermentation rice and then using a freeze-drying process to remove water. Although the water-washing technique is effective in removing the conidia, it necessitates subsequent use of freeze-drying equipment. Therefore, the process take at least 2-3 days per batch. In comparison with the method used in example 3, the water-washing technique is not only time-consuming but also incurs high costs due to the expensive freeze-drying equipment.

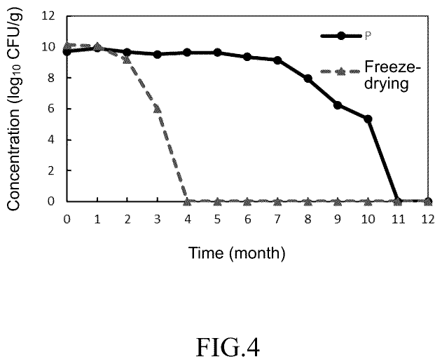

Example 3 involves the use of a thickening agent to adhere to conidia for the preparation of powder. Although the process requires the thickening agent to be added repeatedly 2-3 times, it offers several advantages, including a simplified process flow, the elimination of the need for expensive freeze-drying equipment, time efficiency, and rapid operation. Furthermore, the conidia powder prepared in example 3 has a relatively longer shelf life. As shown in FIG. 4, the conidia powder prepared with Pullulan can be preserved at room temperature for 7 months in which the conidia population remains substantially unchanged. In contrast, the conidia powder prepared through freeze-drying can be preserved at room temperature for only 3 months before the conidia population significantly decreases, becoming undetectable by the end of the 4th month.

1. A method of producing an entomopathogenic fungal conidia powder, comprising:
(a) cultivating the entomopathogenic fungi with a solid culture medium through a solid-state culture to produce conidia;
(b) adding a powder to adhere to the conidia; and
(c) sieving to obtain the conidia powder.

2. The method of claim 1, wherein the solid culture medium comprises rice and soy protein.

3. The method of claim 2, wherein the powder is added when the water content of the solid culture medium is 30-40 wt %.

4. The method of claim 1, wherein the temperature of the solid-state culture is 26-32° C.

5. The method of claim 1, wherein the solid-state culture is tray-style solid-state fermentation.

6. The method of claim 1, wherein the sieving is performed with a sieve with a mesh size of 14 to 30.

7. The method of claim 1, wherein the powder is 5-40% (w/w) of the weight of the solid culture medium.

8. The method of claim 1, wherein a step of drying the conidia is excluded.

9. An entomopathogenic fungal conidia powder, comprising a powder and conidia attached to a surface of the powder.

10. The entomopathogenic fungal conidia powder of claim 9, wherein the entomopathogenic fungi comprise Beauveria spp., Metarhizium anisopliae, Metarhizium rileyi, Isaria javanica, and Purpureocillium takamizusanense.

11. The entomopathogenic fungal conidia powder of claim 9, wherein the powder comprises a thickening agent.

12. The entomopathogenic fungal conidia powder of claim 11, wherein the thickening agent comprises Gum Arabic, Citrus Pectin, Hydroxypropyl Starch, Sodium Polyacrylate, Carrageenan, Gellan Gum, Hydroxyethyl Cellulose, Pullulan, Carboxymethyl Cellulose, Methylcellulose, Xanthan Gum, Guar Gum, and Sodium Alginate.

13. The entomopathogenic fungal conidia powder of claim 9, produced by the method of claim 1.
